(19) United States
(12) Patent Application Publication
(10) Pub. No.: US 2025/0122287 A1
(43) Pub. Date: Apr. 17, 2025

(54) IGG1 FC MONOMER AND APPLICATION THEREOF

(71) Applicant: Suzhou Forlong Biotechnology Co., Ltd., Jiangsu (CN)

(72) Inventors: Tianlei YING, Shanghai (CN); Chunyu WANG, Shanghai (CN)

(21) Appl. No.: 18/939,222

(22) Filed: Nov. 6, 2024

Related U.S. Application Data
(62) Division of application No. 16/759,083, filed on Apr. 24, 2020, now Pat. No. 12,168,689.

(30) Foreign Application Priority Data
Oct. 26, 2017 (CN) 201711014953.3

(51) Int. Cl. C07K 16/28; A61P 35/00; C07K 16/08; C07K 16/12; C07K 16/30; C12N 15/63; C40B 40/10

(52) U.S. Cl. CPC C07K 16/283; C07K 16/08; C07K 16/12; C07K 16/30; C12N 15/63; C40B 40/10; A61P 35/00; C07K 2317/524; C07K 2317/526; C07K 2317/565; C07K 2319/55

(57) ABSTRACT

An IgG1 Fc monomer, a preparation method therefor and an application thereof. Causing an Fc dimer of a novel IgG1 Fc monomer sequence for which an antibody IgG1 constant region is modified to become an Fc monomer by means of the modification on a human antibody IgG1 constant region Fc that uses antibody engineering technology, and maintaining an FcRn binding function; the present application has the feature of very low non-specific binding with unrelated proteins, and the main features of the Fc monomer comprise the T366, L368, P395, and K409 positions in a CH3 region of the constant region of the antibody having mutations, and the monomer being highly efficiently expressed in prokaryotic cells; the monomer may bind to FcRn by using a pH-dependent specific binding mode, and has the feature of very low non-specific binding. By using said Fc monomer, the same may be fused or coupled with various proteins, polypeptides, small molecules, nucleic acids and the like for different targets such that the fused or coupled molecules have the feature of being capable of binding to FcRn by using pH dependence.

### CROSS REFERENCE TO RELATED APPLICATION

This application is a divisional application of U.S. application Ser. No. 16/759,083 filed Apr. 24, 2020, which is a National Phase of PCT Application No. PCT/CN2018/111577 filed Oct. 24, 2018 which claims priority to Chinese Application No. 201711014953.3 filed on Oct. 26, 2017, entitled “Igg1 Fc Monomer and Application Thereof”, each of which is incorporated herein by reference in its entirety.

### SEQUENCE LISTING

The contents of the electronic sequence listing (069777-5US2 Sequence Listing.xml; Size: 6,940 bytes; and Date of Creation: Nov. 6, 2024) is herein incorporated by reference in its entirety.

### TECHNICAL FIELD

The application relates to the technical field of biology, and particularly an IgG1 Fc monomer, a preparation method thereof and an application thereof.

### BACKGROUND

Research shows that there are mainly three long-acting technologies of the current protein drug: PEG (polyethylene glycol) modification technology, HSA (human serum albumin) fusion technology and Fc (human antibody Fc region) fusion technology, which have their own weaknesses respectively, wherein a common key disadvantage is that there is a significant increase in the molecular weight of the fused or modified protein drug, and a significantly decrease in the yield and clinical efficacy of the fused protein drug, for example, human antibody constant region Fc is a homodimer, which has a molecular weight of 60 kDa, and the fusion protein can only be in the dimerized form, which tends to increase the molecular weight of the fusion protein by several times, and meanwhile the dimerized drug molecules may interfere with one another to affect the drug effect. For a long time, researchers have been exploring the use of “antibody fragment” with a smaller molecular weight and soluble expression in prokaryotic cells, as a new generation of antibody drugs with low production costs and strong tissue penetration capabilities. The antibody fragment includes Fab, single-chain antibody (scFv), nanobody, and the like. In order to reduce molecular weight, the antibody fragments need to discard the Fc region of IgG so that they cannot bind to FcRn, resulting in an extremely short half-life (within two hours) in vivo so that it is difficult for the current small molecular weight antibody fragment to be truly applied to clinical treatment of diseases as drugs.

The prior art discloses that the Fc region of human IgG is a homodimer. In the early study of the application, the human IgG1 Fc region is modified by using a novel method of multi-functional screening, and one billion (10⁹) different Fc mutant molecules are screened for druggability and FcRn binding activity, etc., an Fc monomer molecule (mFc) is constructed, which has a molecular weight only half that of the IgG1 Fc region. It completely reserves the FcRn binding property and Protein A/G binding property of the antibody Fc region, and has an excellent property of highly soluble expression in E. coli, the half-life can reach about two days in the animal body; during the research, it is found that it has a very strong non-specific binding property, which may make mFc non-specifically bind to unrelated proteins when used in vivo, greatly limiting its clinical use scope. Therefore, based on the previous research, the present application changes mutation sites and optimizes a screening step (as shown in FIG. 1), obtaining a novel IgG1 Fc monomer and sFc (small-sized Fc) which not only maintains the excellent properties of the previous Fc monomer, but significantly improves non-specific binding property.

The prior art related to the present application is as follows:
[1] HOLLIGER P, HUDSON P J Engineered antibodies fragments and the rise of single domains [J]. Nat: Biotechnol, 2005, 23 (9): 1126-1136.
[2] SAERENS D, GHASSABEH G H, MUYLDERMANS S. Single-domain antibodies as building blocks for novel therapeutics [J]. Curr Opin Pharmacol. 2008, 8 (5): 600-608.
[3] YING T, FENG Y, WANG Y, et al. Monomeric IgG1 Fc molecules displaying unique Fc receptor interactions that are exploitable to treat inflammation-mediated diseases [J]. MAbs, 2014, 6 (5): 1201-1210.
[4] YING T. CHEN W, GONG R, et al. Soluble monomeric IgG1 Fc [J]. J Biol Chem, 2012, 287 (23): 19399-19408.

### SUMMARY

The purpose of the present application is to provide a novel IgG1 Fc monomer having low non-specificity subjected to antibody engineering modification based on the existing technology, and specifically relate to an IgG1 Fc monomer and the application thereof.

Specifically, the present application discloses nucleic acids encoding the IgG1 Fc monomer; some embodiments disclose vectors comprising the nucleic acids, and host cells comprising these vectors.

The IgG1 constant region Fc is a homodimer, it can bind to FcRn (neonatal Fc receptor) with a special pH-dependent binding mode, so that IgG1 has a longer half-life in vivo; the present application is directed to the novel IgG1 Fc monomer sequence after an antibody IgG1 constant region is modified; an Fc dimer becomes an Fc monomer by modification of the human antibody IgG1 constant region Fc using antibody engineering technology, thus maintaining an FcRn binding function and having the feature of very low non-specific binding to unrelated proteins, and the main features of the Fc monomer lie in that the T366, L368, P395, and K409 positions in a CH3 region of the antibody constant region have mutations, and the monomer is highly efficiently expressed in prokaryotic cells; the monomer may bind to FcRn by using a pH-dependent specific binding mode, and has the feature of very low non-specific binding.

Said novel Fc monomer prepared according to the present application may be fused or coupled with various proteins, polypeptides, small molecules, nucleic acids and the like for different targets such that the fused or coupled molecules have the feature of being capable of binding to FcRn by using pH dependence.

The objective of the present application is achieved by the following technical solutions:

### 1. Screening and Judgment of a Novel IgG1 Fc Monomer (sFc) (as Shown in FIG. 1)

In the present application, mutations are performed in the IgG1 constant region and comprise four specific mutation sites and one random mutation site (Leu-351, Thr-366, Leu-368, Pro-395, Lys-409) involved in monomer formation, and further comprise two random mutation sites (Met-428, Asn-434) related to FcRn binding, based on which an IgG1 Fc mutant antibody library with a library capacity of 1.28× 10⁵ is constructed.

A novel IgG1 Fc monomer is screened by biotin-labeled soluble FcRn protein. Biotin-labeled soluble FcRn is immobilized on streptavidin-coated magnetic beads. 10¹² phages displayed Fc at room temperature is incubated with protein G at 1st, 2nd round; and respectively incubated with 5, 4, 2 micrograms of FcRn antigen for two hours at 3rd, 4th, and 5th rounds, 10¹² phages are used for each round of screening, and polyclonal phage ELISA is used to detect enrichment of antibodies. Phages and coated proteins are incubated at the 3rd, 4th, and 5th round, and phage-protein binding is detected with anti-phage HRP-coupled antibodies. Based on polyclonal phage ELISA results, a very significant enrichment is obtained after 4th and 5th round of screening.

In the present application, the phages obtained from these two rounds of screening are used to infect TG1 cells, randomly selecting clones for performing monoclonal phage ELISA, thus further sequencing to identify the enriched IgG1 Fc, and SEC is used to perform monomer judgment. The results are shown in FIG. 1, sFc and another screened protein 1-B10-9 (M428L mutation additionally occurs compared with sFc) are monomers.

### 2. Novel IgG1 Fc Monomer (sFc) Stability Test

In the present application, sFc, 1-B10-9, mFc and Fc are respectively diluted at a concentration of 0.25 mg/ml, and the stability of the proteins is detected at 216 nm by using the instrument Jasco J-815 spectropolarimeter (Jasco International), and the results are shown in FIG. 2: the Tm values of sFc, 1-B10-9, mFc and Fc are: 62.4±0.1° C., 64.0±0.1° C., 58.4±0.2° C., 80.6±0.3° C.

### 3. Binding Capacity of the Novel IgG1 Fc Monomer (sFc) to FcRn by Using Surface Plasmon Resonance (SPR) Detection

In the present application, SA (biotin avidin) chip is used to detect the binding capacity of the novel IgG1 Fc monomer to FcRn, wherein the soluble expression preparation of the novel IgG1 Fc monomer is carried out basically based on the literature. During the detection process, the protein is diluted based on a two-fold ratio (IgG1 Fc dilution 200 nM to 6.25 nM, IgG1 Fc monomer dilution 400 nM to 12.5 nM), the affinity constant is detected by detecting binding and dissociation at pH 6.0, and the results are shown in FIG. 3: the binding capacity to FcRn of the novel IgG1 Fc monomers sFc or 1-B10-9 remains substantially consistent as compared to Fc or mFc.

### 4. Non-Specific Binding Detection of the Novel IgG1 Fc Monomer (sFc)

In the present application, Fc is eukaryotically expressed, mFc and the novel IgG1 Fc monomer sFc are prokaryotically expressed, followed by Protein G purification, and then viruses and cancer-related proteins (gp140, mesothelin, ZIKV EDII, ZIKV EDIII, 5T4, PD-L1, OX40, TIM-3) are used to coat plate (#3690) with 100 ng per well at 4° C. overnight, 100 μl of 3% MPBS (PBS+3% milk) is added into each well, blocking at 37° C. for 1 h, then 0.05% PBST (PBS+0.05% Tween 20) is used for washing, Fc, mFc, novel IgG1 Fc monomer sFc and 1-B10-9 are diluted by 3-fold dilution from 2 μM, incubated with the antigen for 1.5 h at 37° C., washed with 0.05% PBST and added with 1:1000 anti-FLAG-HRP and incubated for 45 minutes at 37° C., and ABTS is added to develop a color at an absorbance of 405 nm. Comparing their non-specific binding capability, the novel IgG1 Fc monomer is significantly more excellent than mFc. The ELISA results shown in FIG. 4 are that the novel IgG1 Fc monomer sFc and 1-B10-9 have a low non-specific binding.

In the present application, the peptide chain of IgG1 Fc monomer comprises CH2 and CH3 domains, wherein the peptide chain comprises an amino acid sequence of SEQ ID NO: 1, X1 is R/T; X2 is L/H; X3 is P/K; X4 is K/T, and Fc is a monomer and capable of binding to a neonatal Fc receptor (FcRn).

The IgG1 Fc monomer peptide comprises an amino acid sequence of SEQ ID NO: 2, wherein X1 is R; X2 is H; X3 is K; X4 is T.

In the present application, the CH3 domain comprises:
(a) amino acid sites 341-447 in the amino acid sequence listed in SEQ ID NO: 1; and/or
(b) amino acid sites 341-447 in the amino acid sequence listed in SEQ ID NO: 2;

The present application provides a fusion protein comprising the above-mentioned IgG1 Fc monomer polypeptide, or the CH3 domain, and a heterologous protein;

The heterologous protein comprises a heavy chain variable region and a light chain variable region, and can specifically bind to an antigen of interest; the heterologous protein described in the embodiment of the present application is an antigen derived from a pathogen; the pathogen is a virus or a bacterium, wherein the virus is selected from human immunodeficiency virus (HIV);

The heterologous protein is also selected from a tumor antigen; wherein the tumor is leukemia, lymphoma, multiple myeloma, malignant melanoma, breast cancer, lung cancer, liver cancer, pancreatic cancer, prostate cancer, colon cancer or renal cell carcinoma.

The heterologous protein is further selected from an autoimmune or inflammatory disease antigen.

The fusion protein of the present application comprises a toxin;

In the fusion protein of the present application, the heterologous protein is selected from a cytokine, a soluble receptor, a growth factor or a label.

In the fusion protein of the present application, the heterologous protein is further selected from a human interferon, an erythropoietin, a soluble tumor necrosis factor receptor, CTLA-4, a soluble IL-4 receptor or a factor IX.

The application also provides a nucleic acid molecule encoding the IgG1 Fc monomer polypeptide, the CH3 domain, and/or the fusion protein.

The application also provides a plasmid comprising the nucleic acid molecule.

The application also provides a host cell comprising the plasmid.

The present application also provides a pharmaceutical composition comprising an effective preventive/therapeutic dose of the IgG1 Fc monomer polypeptide, the CH3 domain, or the fusion protein, and a pharmaceutically acceptable carrier.

The application also provides a pharmaceutical composition comprising an effective preventive/therapeutic dose of the nucleic acid molecule or the plasmid, and a pharmaceutically acceptable carrier.

The present application also provides a medicinal composition comprising an effective preventive/therapeutic dose of the IgG1 Fc monomer Fc polypeptide, the CH3 domain, and/or the fusion protein, coupled to a detectable label; wherein the detectable label is a fluorescent label, a radioactive label, an avidin, a biotin, or an enzyme.

The present application also provides a method for treating an autoimmune disease, an inflammatory disease, a neurodegenerative disease, a cancer or a pathogen infection, comprising administering an effective therapeutic dose of the fusion protein, the nucleic acid molecule, or the plasmid to a subject; wherein the subject has been infected with a pathogen, wherein the fusion protein can specifically bind to the pathogen, and wherein the method is used to treat the pathogen in the infected subject, wherein the pathogen is a virus, particularly an immunodeficiency virus.

The application also provides a method for constructing a recombinant monomeric Fc library, comprising:
(a) introducing a mutation into one or more beta chains of the CH2 or CH3 domain of the IgG1 Fc monomer polypeptide; or
(b) replacing a part of the CH2 domain or the CH3 domain with a complementary determining region (CDR) or a functional fragment of a specific binding antigen retained in a heterologous immunoglobulin variable region; or
(c) both;
using the above method to build a library.

The recombinant monomeric Fc library according to the above method comprises CH2 and CH3 domains, and each IgG1 Fc monomer polypeptide in the library:
(a) being in a monomeric form;
(b) having a molecular weight less than 30 kDa;
(c) being capable of binding to neonatal Fc receptors (FcRn);
(d) comprising an amino acid sequence that is at least 95% identical to SEQ ID NO: 1.

In the present application, a library of nucleic acid molecule-encoding monomeric Fc polypeptide comprises CH2 and CH3 domains, and each Fc polypeptide encoded in the library:
(a) being a monomer;
(b) having a molecular weight less than 30 kDa;
(c) being capable of binding to FcRn;
(d) comprising an amino acid sequence that is at least 95% identical to SEQ ID NO: 1.

The novel IgG1 Fc monomer (sFc) provided by the present application can be used as a small-molecular-weight long-acting monomer module and fused with an antibody fragment to construct a novel genetic engineering antibody, which provides an important theoretical basis and solution for breaking through the bottleneck of the development of antibody drugs; or it can be fused or coupled with various proteins, peptides, small molecules, nucleic acids, etc. for different targets, so that the fused or coupled molecules have the characteristics of binding to FcRn by using pH dependence, which has a potentially better druggability and a longer half-life in vivo.

### BRIEF DESCRIPTION OF THE DRAWINGS

FIG. 1 shows comparison of a novel IgG1 Fc monomer (sFc) with the existing monomers Fc (mFc) and IgG1 Fc by using SEC detection.

FIG. 2 shows a novel IgG1 Fc monomer stability test.

FIG. 3 shows a binding capacity of a novel IgG1 Fc monomer (sFc) to FcRn by surface plasmon resonance (SPR) detection.

FIG. 4 shows a non-specific detection of a novel IgG1 Fc monomer (sFc) with comparison of the existing monomers Fc (mFc) and IgG1Fc.

### DETAILED DESCRIPTION

### Example 1

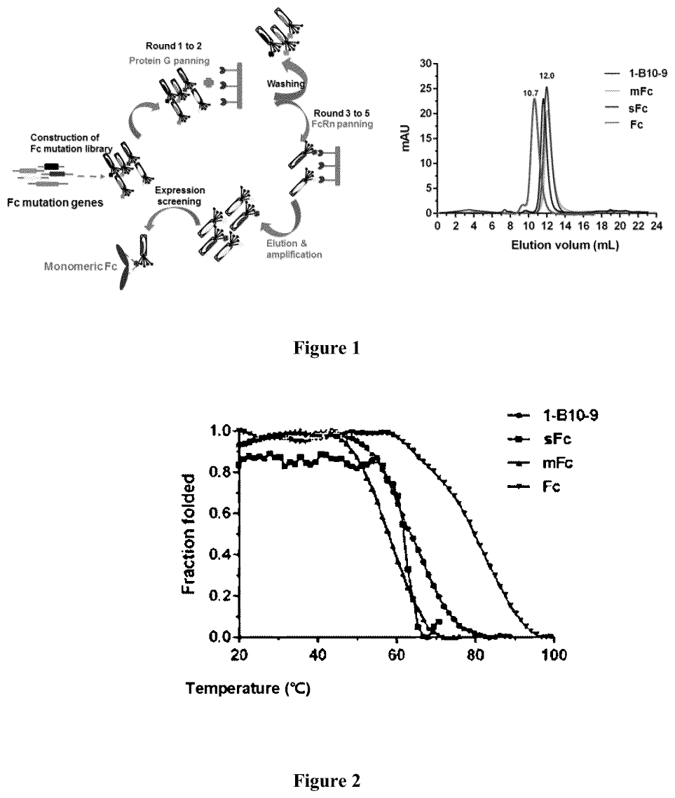

Screening and judgment of IgG1 Fc monomer (sFc): A novel IgG1 Fc monomer is screened by biotin-labeled soluble FcRn protein. Biotin-labeled soluble FcRn is immobilized on streptavidin-coated magnetic beads. 10¹² phages displayed Fc at room temperature is incubated with protein G at 1st, 2nd round; and respectively incubated with 5, 4, 2 micrograms of FcRn antigen for two hours at 3rd, 4th, and 5th rounds, 10¹² phages are used for each round of screening, and polyclonal phage ELISA is used to detect enrichment of antibodies. Phages and coated proteins are incubated at the 3rd, 4th, and 5th round, and phage-protein binding is detected with anti-phage HRP-coupled antibodies. Based on polyclonal phage ELISA results, a very significant enrichment is obtained after 4th and 5th round of screening. The phages obtained from these two rounds of screening are used to infect TG1 cells, randomly selecting clones for performing monoclonal phage ELISA, thus further sequencing to identify the enriched IgG1 Fc, and SEC is used to perform monomer judgment. The results are shown in FIG. 1, sFc and another screened protein 1-B10-9 (M428L mutation additionally occurs compared with sFc) are monomers.

Novel IgG1 Fc monomer (sFc) stability test: In the present application, sFc, 1-B10-9, mFc and Fc are respectively diluted at a concentration of 0.25 mg/ml, and the stability of the proteins is detected at 216 nm by using the instrument Jasco J-815 spectropolarimeter (Jasco International), and the results are shown in FIG. 2: the Tm values of sFc, 1-B10-9, mFc and Fc are: 62.4±0.1° C., 64.0±0.1° C., 58.4±0.2° C., 80.6±0.3° C.

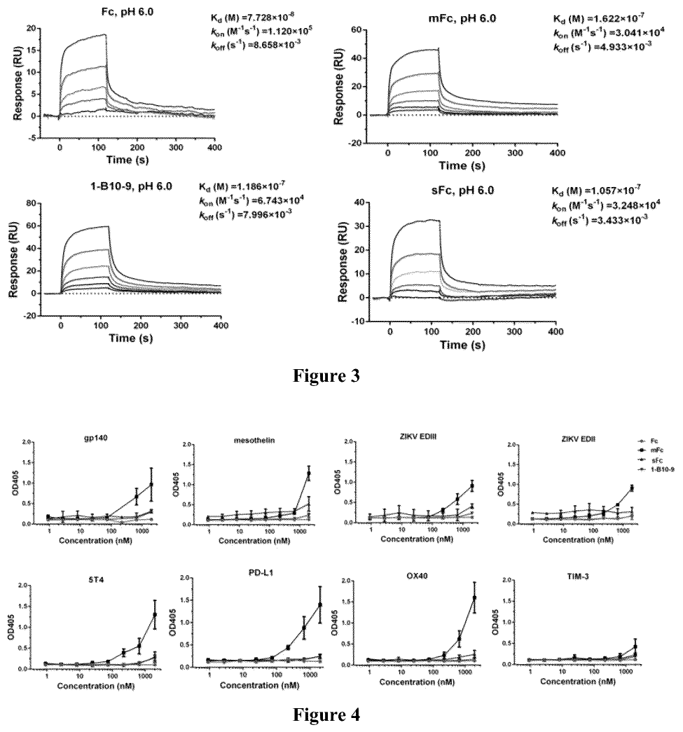

Binding capacity of the novel IgG1 Fc monomer (sFc) to FcRn by using surface plasmon resonance (SPR) detection: In the present application, SA (biotin avidin) chip is used to detect the binding capacity of the novel IgG1 Fc monomer to FcRn, wherein the soluble expression preparation of the novel IgG1 Fc monomer is carried out basically based on the literature. During the detection process, the protein is diluted based on a two-fold ratio (IgG1 Fc dilution 200 nM to 6.25 nM, IgG1 Fc monomer dilution 400 nM to 12.5 nM), the affinity constant is detected by detecting binding and dissociation at pH 6.0, the results are shown in FIG. 3: the binding capacity to FcRn of the novel IgG1 Fc monomers sFc or 1-B10-9 remains substantially consistent as compared to Fc or mFc.

Non-specific binding detection of the novel IgG1 Fc monomer (sFc): In the present application, Fc is eukaryotically expressed, mFc and the novel IgG1 Fc monomer sFc are prokaryotically expressed, followed by Protein G purification, and then viruses and cancer-related proteins (gp140, mesothelin, ZIKV EDII, ZIKV EDIII, 5T4, PD-L1, OX40, TIM-3) are used to coat plate (#3690) with 100 ng per well at 4° C. overnight, 100 μl of 3% MPBS (PBS+3% milk) is added into each well, blocking at 37° C. for 1 h, then 0.05% PBST (PBS+0.05% Tween 20) is used for washing, Fc, mFc, novel IgG1 Fc monomer sFc and 1-B10-9 are diluted by 3-fold dilution from 2 μM, incubated with the antigen for 1.5 h at 37° C., washed with 0.05% PBST and added with 1:1000 anti-FLAG-HRP and incubated for 45 minutes at 37° C., and then ABTS is added to develop a color at an absorbance of 405 nm. Comparing their non-specific binding capability, the novel IgG1 Fc monomer is significantly more excellent than mFc. The ELISA results shown in FIG. 4 are that the novel IgG1 Fc monomer sFc and 1-B10-9 have a low non-specific binding.

What is claimed is:

1. A method for treating a cancer in a subject in need thereof, comprising administering to the subject an effective therapeutic amount of a fusion protein comprising:
(i) an IgG1 Fc monomer polypeptide comprising CH2 and CH3 domains, wherein the polypeptide comprises arginine (R), histidine (H), lysine (K) and threonine (T) at positions 366, 368, 395 and 409 of IgG1, respectively, wherein the numbering is according to EU numbering and the Fc monomer polypeptide is capable of binding to a Fc receptor (FcRn), wherein the binding affinity of the IgG1 Fc monomer polypeptide to at least one antigen selected from the group consisting of 5T4, PD-L1 and OX40 is lower than that of a wild-type IgG1 Fc, as measured by an enzyme-linked immunosorbent assay (ELISA) with 100 ng of the antigen and up to 223 nM of the IgG1 Fc monomer polypeptide; and
(ii) a heterologous protein.

2. The method of claim 1, wherein the IgG1 Fc monomer further comprises an amino acid substitution at position 428 of IgG1.

3. The method of claim 1, wherein said heterologous protein is a tumor antigen.

4. The method of claim 1, wherein the heterologous protein is a cytokine.

5. The method of claim 2, wherein said heterologous protein is a tumor antigen.

6. The method of claim 2, wherein the heterologous protein is a cytokine.

7. The method of claim 1, wherein the cancer is selected from the group consisting of leukemia, lymphoma, multiple myeloma, malignant melanoma, breast cancer, lung cancer, liver cancer, pancreatic cancer, prostate cancer, colon cancer and renal cell carcinoma.

8. A method for treating a cancer in a subject in need thereof, comprising administering to the subject an effective therapeutic amount of a fusion protein comprising:
(i) an IgG1 Fc monomer polypeptide comprising CH2 and CH3 domains, wherein the CH3 domain comprises the amino acid sequence having residues 111 to 217 of SEQ ID NO: 2, which correspond to positions 341 to 447 of IgG1 according to EU numbering, wherein the binding affinity of the IgG1 Fc monomer polypeptide to at least one antigen selected from the group consisting of 5T4, PD-L1 and OX40 is lower than that of a wild-type IgG1 Fc, as measured by an enzyme-linked immunosorbent assay (ELISA) with 100 ng of the antigen and up to 223 nM of the IgG1 Fc monomer polypeptide; and
(ii) a heterologous protein.

9. The method of claim 8, wherein said heterologous protein is a tumor antigen.

10. The method of claim 8, wherein the heterologous protein is a cytokine.

11. The method of claim 8, wherein the cancer is selected from the group consisting of leukemia, lymphoma, multiple myeloma, malignant melanoma, breast cancer, lung cancer, liver cancer, pancreatic cancer, prostate cancer, colon cancer and renal cell carcinoma.

12. A nucleic acid molecule encoding an IgG1 Fc monomer polypeptide comprising CH2 and CH3 domains, wherein the polypeptide comprises arginine (R), histidine (H), lysine (K) and threonine (T) at positions 366, 368, 395 and 409 of IgG1, respectively, wherein the numbering is according to EU numbering and the Fc monomer polypeptide is capable of binding to a Fc receptor (FcRn).

13. A plasmid, comprising the nucleic acid molecule of claim 12.

14. A host cell, comprising the plasmid of claim 13.

15. The nucleic acid molecule of claim 12, encoding a fusion protein comprising the IgG1 Fc monomer polypeptide and a heterologous protein.

16. The nucleic acid molecule of claim 15, wherein the heterologous protein comprises a tumor antigen.

17. The nucleic acid molecule of claim 15, wherein the heterologous protein comprises a cytokine.

18. The nucleic acid molecule of claim 15, wherein the IgG1 Fc monomer polypeptide comprises the amino acid sequence of SEQ ID NO: 2

19. A plasmid, comprising the nucleic acid molecule of claim 15.

20. A host cell, comprising the plasmid of claim 19.


SEQUENCE LISTING (2 sequences)

SEQ ID NO: 1 - protein, 217 aa, synthetic construct
  REGION 1..217: note = Constant region Fc of Antibody
  VAR_SEQ 136: note = Xaa=Arg/Thr
  VAR_SEQ 138: note = Xaa=Leu/His
  VAR_SEQ 165: note = Xaa=Pro/Lys
  VAR_SEQ 179: note = Xaa=Lys/Thr
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLXCXVKGFYPSDIAVEWESNGQPENNYKTTXPVLDSDGSFFLYSXLTVDKSRWQQGNVFSCSVMHEALHNHYTQKSLSLSPGK

SEQ ID NO: 2 - protein, 217 aa, synthetic construct
  REGION 1..217: note = Constnt region Fc of antibody
  VAR_SEQ 136: note = Xaa=Arg
  VAR_SEQ 138: note = Xaa=His
  VAR_SEQ 165: note = Xaa=Lys
  VAR_SEQ 179: note = Xaa=Thr
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVMHEALHNHYTQKSLSLSPGK